(19) United States
(12) Patent Application Publication
(10) Pub. No.: US 2002/0009490 A1
(43) Pub. Date: Jan. 24, 2002

(54) Antiviral therapeutic composition and treatment

(76) Inventors: Travis R. Benkendorfer, Nairobi (KE); Loyd V. Allen JR., Edmond, OK (US)

Correspondence Address: PERKINS, SMITH & COHEN LLP, ONE BEACON STREET, 30TH FLOOR, BOSTON, MA, 02108, US

(21) Appl. No.: 09/747,827

(22) Filed: Dec. 22, 2000

Related U.S. Application Data
(60) Provisional application No. 60/171,697, filed on Dec. 22, 1999.

(51) Int. Cl. A61K 9/20

(52) U.S. Cl. 424/464

(57) ABSTRACT

This invention described herein discloses an antiviral therapeutic composition. This antiviral therapeutic composition can be used to treat human viral infections.

### CROSS-REFERENCE TO RELATED APPLICATIONS

This application claims priority to U.S. Provisional Patent Application No. 60/171,697, entitled, “VIRON, A Therapeutic Composition and Its Use in Treating Human Viral Infections (3),” filed Dec. 22, 1999. This application is a continuation-in-part application of U.S. patent application Ser. No. 08/826,371, entitled, “VIRON, A Therapeutic Composition; Method of Making VIRON; and Its Use in Treating Human Viral Infections,” filed on Apr. 9, 1997, and both its divisional application (U.S. patent application Ser. No. 08/232,686), filed Nov. 5, 1998 and its continuation application (same Ser. No. as the parent), filed Nov. 6, 1998. This application is also a continuation-in-part application of U.S. patent application Ser. No. 08/826,368, entitled, “VIRON, A Therapeutic Composition; Method of Making VIRON; and Its Use in Treating Human Viral Infections,” also filed on Apr. 9, 1997 and its continuation application (same Ser. No. as the parent), filed Oct. 19, 1998.

### FIELD OF THE INVENTION

This invention discloses a therapeutic composition that can be used to treat human viral infections as well as methods for treating viral infections with this therapeutic composition.

### BACKGROUND OF THE INVENTION

While the acquired immunodeficiency syndrome (AIDS) epidemic appears to be gaining in North America, Latin America, Western Europe, and Asia, it continues to grow in Africa. Currently, most of the approximately 28 million people worldwide who are infected with human immunodeficiency virus (HIV) reside in developing countries with almost two-thirds of these individuals residing in the sub-Saharan African countries of Zambia, Zimbabwe, Kenya, Uganda, Tanzania, Nigeria, Congo, Ghana and Benin. More women than men in these countries are now infected and almost half of the pregnant women in some cities test positively for the presence of HIV. Programs to change the behaviors in these countries that cause increased risk of infection, such as male promiscuity and low condom use, have been largely unsuccessful. As a result, the great strides made in public health in these countries have been reversed and life expectancy has dropped due to this increasing incidence of HIV infection.

The fact that HIV is capable of undergoing significant mutations complicates the development of effective treatments. While HIV-1 remains the infective strain seen in most of the world, variants have recently appeared. The most significant variant to appear is HIV-2, which was isolated from AIDS patients in West Africa in 1986. Surprisingly, the proteins encoded by HIV-2 are only about 40% homologous to proteins encoded by the original HIV isolates. Dual infection with HIV-1 and HIV-2 is associated with over 63% of the AIDS cases in the Ivory Coast. AIDS patients with this dual infection generally have a life expectancy of less than one year, resulting from the effects of the infection itself and the susceptibility to opportunistic infections, Kaposi's sarcoma, malnutrition, and persistent diarrhea.

A treatment which is effective in delaying, controlling, or reversing the course of AIDS in individuals having combined HIV-1 and HIV-2 infections would be very desirable. An effective treatment for individuals having combined HIV-1 and HIV-2 infections would prolong their drastically shortened life, thus allowing further treatment of the HIV infection itself and its associated conditions. An effective treatment for individuals having combined HIV-1 and HIV-2 infections could also result in the deadly dual infection epidemic being eradicated or at least confined to its present locations. A treatment which is effective in converting a clinically significant portion of patients who are positive for HIV-1 and HIV-2 to those with a negative status would be a substantial step in finding a cure for AIDS.

### SUMMARY OF THE INVENTION

The invention described herein discloses a composition of matter having anti-viral properties that is designated VIRON. VIRON comprises a mixture of chemicals combined with the biological immunomodulator drug, alpha-interferon. VIRON can be used as a clinical treatment for viral infections, generally.

By using VIRON, the inventors have discovered a new and useful HIV treatment method that increased CD4+ lymphocyte levels in patients infected with HIV in Africa. Using these methods, they have developed an effective treatment for individuals infected by both HIV-1 and HIV-2, thus overcoming the difficulties seen in the prior art. These methods have the potential to lead to improved treatment of AIDS patients and the development of a cure for this disease.

### DETAILED DESCRIPTION OF THE INVENTION

This invention discloses a VIRON base material comprising a mixture of inositol oxidized with nitric acid whose pH is increased by using potassium hydroxide to which sodium sulfite, copper sulfate, pyrocatechol, and benzoquinone are then added after dilution with purified water and heating. The liquid is dried to a powder. Finally, germanium sesquioxide (2-carboxyethyl germanium sesquioxide) and alpha-interferon is added to the VIRON base material to form the therapeutic composition, VIRON.

The compositions of this invention as well as their method of production and use in clinical treatment are further illustrated by the following:

Preparation of VIRON Base

In a preferred embodiment, an approximately 48% solution of inositol purchased from Japan or China in concentrated (approximately 63%) nitric acid is prepared in a 10-gallon stainless steel container with an open lid in a vented exhaust hood. A cover is lightly placed on the container where it is allowed to sit undisturbed for approximately 24 hours until all the nitric acid fumes have evaporated. It may be necessary to gently heat the solution in a water bath to accomplish this step. The mixture appears dark brown at this stage.

Next, approximately 10% potassium hydroxide is added with constant stirring until a pH of at least 4.5 or higher is obtained. The stainless steel container is placed in a hot water bath (approximately 90 degrees C.) for approximately 2 hours after which it is removed and allowed to cool to room temperature.

The stainless steel container is then heated to boiling. While heating, approximately 6 times the volume of purified water is added. When this mixture begins to boil, approximately 12.143% of anhydrous sodium sulfite, approximately 0.00057143% copper sulfate, approximately 2.36% pyrocatechol, and approximately 2.36% benzoquinone are added with constant stirring for 10 to 20 minutes until the added chemicals have all dissolved. Additional purified water is added, if required, to final volume. The container is removed from the heat and allowed to stand until it has cooled to room temperature.

Preparation of VIRON Powder

The VIRON liquid is poured into large surface area containers where it is allowed to dry to a dark brown powder. Gentle heat may be used. The resulting powder is pulverized and sieved in preparation for the tablet blend.

Preparation of VIRON Tablets

In a preferred embodiment, VIRON tablets can be prepared in either sintered or compressed tablet forms. For sintered tablets, the VIRON powder, germanium sesquioxide, citric acid, sodium bicarbonate, polyethylene glycol 3350 and flavor are weighed and blended together until uniform. VIRON tablet blend is then placed in a plastic blister mold and gently tamped. The plastic blister mold containing the powder is placed in an oven at approximately 90 degrees C. for 10 to 12 minutes for the sintering process to occur. The mold is removed from the oven and placed in a refrigerator for approximately 5 minutes. Then, approximately 50 microliters of a solution of the human alpha-interferon is dropped onto the tablet and allowed to dry under gentle moving air. The mold containing the formed tablet is removed and the final sheet-seal is affixed onto the plastic blister.

For compressed tablets, a solution of human alpha-interferon is blended with the corn starch, spread out and allowed to air-dry under gentle moving air. VIRON powder, germanium sesquioxide, citric acid, sodium bicarbonate, polyethylene glycol 3350, xylitol, the corn starch:human alpha-interferon mixture, sodium lauryl sulfate, and powdered flavor are weighed and blended together until uniform. The VIRON tablet, powder blend is then placed in a tableting machine and the required adjustments are made for a tablet of the proper weight and hardness. The tablets are compressed and packaged.

Component Ratios

In a preferred embodiment, each sintered tablet contains approximately 533 milligrams of VIRON powder, approximately 333 milligrams of germanium sesquioxide, approximately 25 International Units of human alpha-interferon, approximately 0.05 to 1 milligram of pyrocatechol, approximately 0.05 to 1 milligram of benzoquinone, approximately 5 to 25 milligrams of citric acid, approximately 5 to 25 milligrams of sodium bicarbonate, approximately 200 to 400 milligrams of polyethylene glycol, and approximately 25 to 75 milligrams of flavor. Compressed tablets additionally contain approximately 25 to 75 milligrams of xylitol, approximately 10 to 50 milligrams of corn starch, and approximately 5 to 25 milligrams of sodium lauryl sulfate. Other non-tablet formulations of VIRON are envisioned within this invention as long as these component ratios are maintained.

Treatment Regimens

Treatment regimens for VIRON are determined by patient response. The frequency and interval of treatment is determined by the results of state of the art tests for measuring viral disease progression, such as the measurement of plasma viral loads. Treatment regimens can also be varied throughout the administration of VIRON in a further attempt at maximizing patient response.

Variations

The ratios of the components in VIRON can be varied within the ranges indicated. While the preferred VIRON formulation is a tablet, any pharmaceutically acceptable carrier and delivery method can be used, including, but not limited to, liquid, gel, oral, injectable, and suppository forms. Similarly, the dosage schedule can be varied at will with the only two limiting parameters being efficacy in treating viral infection and non-toxicity to the treated patient. Efficacy of treatment can be measured by any suitable state of the art method, including, but not limited to, viral loads, CD4+ T cell levels, and amelioration of symptoms associated with the viral infection.

It will now be apparent to those skilled in the art that other embodiments, improvements, details, and uses can be made consistent with the letter and spirit of the foregoing disclosure and within the scope of this patent, which is limited only by the following claims, construed in accordance with the patent law, including the doctrine of equivalents.

What is claimed is:

1. An antiviral therapeutic composition comprising:
(a) approximately 533 milligrams of VIRON powder;
(b) approximately 333 milligrams of germanium sesquioxide;
(c) approximately 25 International Units of human alpha-interferon;
(d) approximately 0.05 to 1 milligram of pyrocatecol;
(e) approximately 0.05 to 1 milligram of benzoquinone;
(f) approximately 5 to 25 milligrams of citric acid;
(g) approximately 5 to 25 milligrams of sodium bicarbonate;
(h) approximately 200 to 400 milligrams of polyethylene glycol; and
(i) approximately 25 to 75 milligrams of flavor.

2. The antiviral therapeutic composition of claim 1 wherein said composition is mixed with a pharmaceutically acceptable carrier to make a suitable therapeutically active dosage formulation.

3. The antiviral therapeutic composition of claim 2 wherein said dosage formulation is a tablet.

4. The antiviral therapeutic composition of claim 3 wherein said tablet is sintered.

5. The antiviral therapeutic composition of claim 1 further comprising:
(a) approximately 25 to 75 milligrams of xylitol;
(b) approximately 10 to 50 milligrams of corn starch; and
(c) approximately 5 to 25 milligrams of sodium lauryl sulfate.

6. The antiviral therapeutic composition of claim 5 wherein said composition is mixed with a pharmaceutically acceptable carrier to make a suitable therapeutically active dosage formulation.

7. The antiviral therapeutic composition of claim 6 wherein said dosage formulation is a tablet.

8. The antiviral therapeutic composition of claim 7 wherein said tablet is compressed.

9. A method of preparing VIRON base comprising:
(a) mixing an approximately 48% solution of inositol in concentrated nitric acid in a stainless steel container with an open lid in a vented exhaust hood;
(b) allowing said inositol solution to sit lightly covered for approximately 24 hours until all the nitric acid fumes have evaporated with the optional aid of gentle heating in a water bath;
(c) adding approximately 10% potassium hydroxide to said inositol solution with constant stirring until a pH of at least 4.5 or higher is obtained;
(d) heating said inositol solution in said stainless steel container in an approximately 90 degree C. hot water bath for approximately 2 hours;
(e) allowing said inositol solution to cool to room temperature;
(f) heating said inositol solution to boiling while adding approximately 6 times the volume of purified water;
(g) adding approximately 12.143% of anhydrous sodium sulfite, approximately 0.00057143% copper sulfate, approximately 2.36% pyrocatechol, and approximately 2.36% benzoquinone once said inositol solution is boiling using constant stirring for 10 to 20 minutes until the added chemicals have all dissolved; and
(h) allowing said inositol solution to stand until it has cooled to room temperature.

10. A method of preparing VIRON powder comprising:
(a) pouring VIRON liquid into large surface area containers;
(b) allowing said VIRON liquid to dry to a dark brown powder, optionally using gentle heat; and
(c) pulverizing and sieving said VIRON powder.

11. A method of preparing sintered VIRON tablets comprising:
(a) blending VIRON powder, germanium sesquioxide, citric acid, sodium bicarbonate, polyethylene glycol 3350 and flavor together until uniform;
(b) placing VIRON tablet blend in a plastic blister mold;
(c) tamping said VIRON tablet blend gently;
(d) heating a plastic blister mold containing said VIRON tablet blend in an oven at approximately 90 degrees C. for 10 to 12 minutes for the sintering process to occur;
(e) removing said plastic blister mold from the oven;
(f) placing said plastic blister mold in a refrigerator for approximately 5 minutes;
(g) dropping approximately 50 microliters of a solution of the human alpha-interferon onto the formed tablet; and
(h) allowing said formed tablet to dry under gentle moving air.

12. A method of preparing sintered VIRON tablets comprising:
(a) blending a solution of human alpha-interferon with the corn starch;
(b) spreading out the corn starch:human alpha-interferon combination;
(c) allowing said combination to air-dry under gentle moving air;
(d) blending VIRON powder, germanium sesquioxide, citric acid, sodium bicarbonate, polyethylene glycol 3350, xylitol, the corn starch:human alpha-interferon mixture, sodium lauryl sulfate, and powdered flavor until uniform;
(e) placing the tablet blend in a tableting machine; and
(f) compressing the tablet blend into tablets.

13. A method of treating a viral infection comprising administering VIRON to an infected patient.

14. The method of claim 13 wherein said viral infection is caused by human immunodeficiency virus.

15. The method of claim 13 wherein said patient has acquired immunodeficiency syndrome.

16. A method of decreasing the amount of virus in biological samples.

17. The method of claim 16 wherein said biological sample is human plasma.

18. A method of increasing the amount of CD4+ T cells in biological samples.

19. The method of claim 18 wherein said biological sample is human plasma.
